(19) United States
(12) Patent Application Publication
(10) Pub. No.: US 2022/0411467 A1
(43) Pub. Date: Dec. 29, 2022

(54) PROTEIN BIOPROCESS

(71) Applicant: NUTRITION & BIOSCIENCES USA 1, LLC, Rochester, PA (US)

(72) Inventors: Joshua Stephen Katz, Wilmington, DE (US); Susan Lynn Jordan, Wilmington, DE (US)

(21) Appl. No.: 17/784,396

(22) PCT Filed: Dec. 8, 2020
(86) PCT No.: PCT/US2020/063720

§ 371 Date: Jun. 10, 2022

Related U.S. Application Data
(60) Provisional application No. 62/947,175, filed on Dec. 12, 2019.

(51) Int. Cl. C07K 1/34; B01D 61/14; B01D 69/02

(52) U.S. Cl. CPC C07K 1/34; B01D 61/145; B01D 69/02; B01D 2315/16; B01D 2325/34; B01D 2317/02

(57) ABSTRACT

This disclosure relates to a method which involves the steps of: (a) providing an aqueous solution comprising a protein and a polyalkoxy fatty acyl surfactant of general formula I

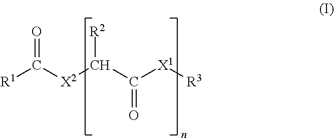

wherein R¹—C(═O) is a fatty acyl group, R² is H or a substituted or unsubstituted hydrocarbyl group, X¹ is S, O or NH, X² is S, O or NH, n is 0 or an integer of 1-5, R³ is a polymeric group comprising polymerized units of general formula II and III

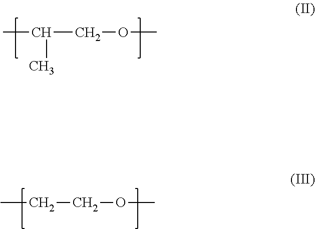

(b) contacting the aqueous solution with a separation membrane, and (c) subjecting the aqueous solution to a diafiltration step and/or to an ultrafiltration step to produce a retentate product which is an aqueous solution comprising the protein, whereby the compound of formula I reduces aggregation of the protein in method steps (a)-(c) and whereby the compound of formula I passes through the separation membrane in step (c).

### BACKGROUND

### Field of the Disclosure

The present disclosure relates to a method of stabilizing a protein in aqueous solution during purification and/or concentration of the protein, in particular during ultrafiltration and/or diafiltration procedures.

### Description of Related Art

Biologics, pharmaceuticals derived from proteins or other biologically-derived macromolecules, have rapidly emerged as an important class of pharmaceuticals. Due to the relatively fragile nature of protein materials, development of biologic actives that are both therapeutically beneficial and sufficiently stable to withstand processing, distribution, and administration remains a significant challenge. Protein biologic drug producers and formulators employ a variety of process techniques to purify and prepare the biologic for its final dosage form. These include ultrafiltration (UF) and diafiltration (DF) procedures that are frequently employed to purify and concentrate proteins or exchange buffer solutions. However, ultrafiltration and diafiltration can also cause protein denaturation, aggregation, or precipitation due to shear stress, contact between the protein and an interface, or higher protein concentration at the filtration membrane surface during ultrafiltration or diafiltration operations.

Surfactants are commonly used in final formulations of protein-based biologics to protect the biologic from various destabilizing forces such as interfaces and shear. While such forces are also present earlier in the development of the biologic formulation (such as during upstream and downstream processing), use of surfactants is limited due to interaction of the surfactant with processing equipment (e.g., fouling of membranes) and difficulty in reliably removing the surfactant during protein filtration, buffer exchange, and concentration.

Polysorbate 80 is the most commonly used surfactant in biologic formulations. Callahan, Stanley, and Li, JOURNAL OF PHARMACEUTICAL SCIENCES 103:862-869, 2014, demonstrate the ability of polysorbate 80 to stabilize protein during an ultrafiltration and diafiltration process, but also state that polysorbate 80 is concentrated and does not effectively pass through an ultrafiltration and diafiltration filter.

It is stated in a website (https://www.researchgate.net/post/How_to_add_TWEEN 80_after protein_ultrafiltration_diafiltration) that Tween 80 (polysorbate 80) is adsorbed to membranes and therefore should be added after diafiltration.

US20130195888A1 teaches and exemplifies using polysorbate 80 in a diafiltration process at a concentration of 0.1 mg/mL. While the protein is protected from aggregation, there is no evaluation of the change in polysorbate 80 content during the ultrafiltration concentration step.

Lei et al, Biotechnol. Prog., 2013, Vol. 29, No. 6 demonstrate that polysorbate 20 composition and concentration changes when using a 30 kDa (molecular weight cut-off) ultrafiltration membrane. The membrane retains polysorbate 20 regardless of whether or not the concentration is above or below the critical micelle concentration of polysorbate 20.

Poloxamer 188 is used frequently in upstream bioprocessing as a stabilizer for cells producing the biologic. However, the poloxamer is removed upstream of the ultrafiltration and diafiltration process during Protein A chromatography, and is therefore not present to stabilize the biologic through downstream processing (such as ultrafiltration and diafiltration). An example where this is shown is in Xu, et al. Bioprocess Biosyst Eng (2017) 40:1317-1326.

### BRIEF SUMMARY OF THE DISCLOSURE

The present disclosure provides a method of stabilizing a protein in aqueous solution during purification and/or concentration of the protein. The method comprises the steps of: (a) providing an aqueous solution comprising a protein and a polyalkoxy fatty acyl surfactant of general formula I

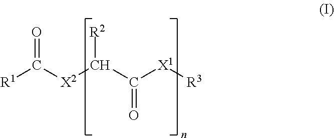

wherein R¹—C(═O) is a fatty acyl group, R² is H or a substituted or unsubstituted hydrocarbyl group, X¹ is S, O or NH, X² is S, O or NH, n is 0 or an integer of 1-5, R³ is a polymeric group comprising polymerized units of general formula II and III

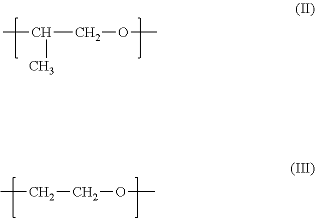

(b) contacting the aqueous solution with a separation membrane, and (c) subjecting the aqueous solution to a diafiltration step to reduce the concentration of soluble low molecular weight components or to introduce one or more soluble low molecular weight components therein and/or to an ultrafiltration step so as to concentrate the protein to produce a retentate product which is an aqueous solution comprising the protein, whereby the compound of formula I reduces aggregation of the protein in method steps (a)-(c) and whereby the compound of formula I passes through the separation membrane in step (c).

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

While compositions and methods are described herein in terms of “comprising” various components or steps, the compositions and methods also can “consist essentially of” or “consist of” the various components or steps, unless stated otherwise.

Before addressing details of embodiments described below, some terms are defined or clarified.

The term “aqueous solution”, as used herein, means a solution in which the solvent comprises at least 90 wt % of water based on the total weight of the solvent. In some embodiments, the solvent further comprises an organic solvent such as acetone, ethanol, DMSO (dimethyl sulfoxide), 2-butanone, ethyl caprylate and ethyl laurate. In some embodiments, the solvent comprises, consists essentially of or consists of water and an organic solvent. In some embodiments, the solvent comprises at least 92 wt %, or at least 94 wt %, or at least 96 wt %, or at least 98 wt %, or at least 99 wt % of water based on the total weight of the solvent. In some embodiments, the solvent consists essentially of or consists of water. In some embodiments, the solvent is water. In some embodiments, the aqueous solution is substantially free of an organic solvent. In some embodiments, the liquid medium of the aqueous solution consists essentially of or consists of water.

The term “ultrafiltration”, as used herein, means a process in which a solution of a protein is passed through a semipermeable membrane (i.e., separation membrane) that retains the protein (the retentate) while permitting the solvent and dissolved low molecular weight components such as salts and sugars (the filtrate) to pass through. In this way, the protein solution becomes more concentrated.

The term “diafiltration”, as used herein, means a filtration and solvent exchange process in which a solution of a protein is filtered by using a semipermeable membrane (i.e., separation membrane) that retains the protein (the retentate) while permitting a portion of the solvent and dissolved low molecular weight components such as salts and sugars (the filtrate) to pass through. The lost solvent (i.e., solvent passing through separation membrane) is replaced by a new solvent which optionally contains new low molecular weight component(s) dissolved therein, and the resulting new solution is subjected to filtration again by using a semipermeable membrane (i.e., separation membrane) that retains the protein (the retentate) while permitting a portion of the solvent and dissolved low molecular weight components such as salts and sugars (the filtrate) to pass through.

The diafiltration can be conducted continuously or in batch. In some embodiments, the diafiltration is conducted in continuous mode and a new solvent is continuously added to the retentate at the same rate as the filtrate is generated. In such embodiments, the concentration of dissolved low molecular weight components such as salts or sugars in a solution of a protein can be reduced without substantially changing the concentration of the protein in the solution. In some embodiments, the new solvent comprises low molecular weight components such as salts and sugars dissolved therein which are different from the low molecular weight components contained in the original solution of a protein. In such embodiments, diafiltration can be used to introduce one or more low molecular weight components in the protein solution.

The term “separation membrane”, as used herein, means a porous membrane or filter that is used in the ultrafiltration (UF) or diafiltration (DF) to separate components in the aqueous solution based on their molecular weight or size. Molecules such as proteins that are larger than the pores in the membrane are retained and low molecular weight compounds that are smaller than the pores pass through.

The term “surfactant/protein concentration ratio”, as used herein, means the ratio of the concentration of polyalkoxy fatty acyl surfactant of formula I to the concentration of protein in an aqueous solution. The concentration of polyalkoxy fatty acyl surfactant of formula I and the concentration of protein are expressed as weight volume ratio (e.g., mg/ml) in the present disclosure.

A polyalkoxy compound is a compound that contains one or more group having the structure -(-A-O)ₘ—, where m is three or more, and A is an unsubstituted alkyl group. The group A may be linear, branched, cyclic, or a combination thereof. The various A groups among the various -(-A-O)— groups may be the same as each other or different.

A fatty compound is a compound that contains one or more fatty group. A fatty group is a group that contains 8 or more carbon atoms, each of which is bonded to one or more of the other carbon atoms in the group. A polyalkoxy fatty compound is a compound that is both a polyalkoxy compound and a fatty compound.

Number-average molecular weight is defined as the total weight of a sample divided by the number of molecules in the sample.

A hydrocarbyl group is a group that contains hydrogen and carbon atoms. An unsubstituted hydrocarbyl group contains only hydrogen and carbon atoms. A substituted hydrocarbyl group contains one or more substituent group that contains one or more atom other than hydrogen and carbon.

A protein is a polymer in which the polymerized units are amino acids. The amino acids are bonded together by peptide bonds. A protein contains 20 or more polymerized units of one or more amino acid residues. The term protein includes linear polypeptide chains as well as more complex structures that contain polypeptide chains.

A protein is considered to be in solution in a liquid medium (or, synonymously, dissolved in the liquid medium) if the molecules of the protein are distributed throughout the continuous liquid medium in the form of dissolved individual molecules. The protein is considered to be dissolved in water if the continuous liquid medium contains water in the amount of 60% or more by weight based on the weight of the continuous liquid medium.

A chemical group is an ionic group if there is a pH value between 4.5 and 8.5 such that, when the chemical group is in contact with water at that pH value, 50 mole % or more of those chemical groups present are in ionic form.

A buffer is either (i) a compound that has the ability to accept a proton to form the conjugate acid of that compound, and the conjugate acid of that compound has pKa of less than 10, or (ii) a compound that has the ability to release a proton, and the compound has pKa of greater than 4.

It has now surprisingly been found that a certain class of surfactant compounds that has previously been found to stabilize proteins in aqueous formulations as disclosed in WO2017/044367, which is incorporated herein by reference in its entirety for all purposes, can be efficiently and effectively removed from protein solutions when these are subjected to ultrafiltration and/or diafiltration processes. This permits the use of surfactants in this class at upstream stages of the production of the proteins (rather than just at the formulation stage), where they can mitigate protein aggregation and particle formation taking place during purification and/or concentration of the protein solutions, thereby improving protein yields. Unlike polysorbates, surfactants of this class are readily filtered through separation membranes at concentrations that are still protective of proteins and therefore the problem of protein denaturation and aggregation during filtration can be mitigated.

Accordingly, the present disclosure provides a method of stabilizing a protein in aqueous solution during purification and/or concentration of the protein. The method comprises the steps of: (a) providing an aqueous solution comprising a protein and a polyalkoxy fatty acyl surfactant of general formula I

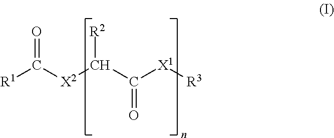

wherein R¹—C(═O) is a fatty acyl group, R² is H or a substituted or unsubstituted hydrocarbyl group, X¹ is S, O or NH, X² is S, O or NH, n is 0 or an integer of 1-5, R³ is a polymeric group comprising polymerized units of general formula II and Ill

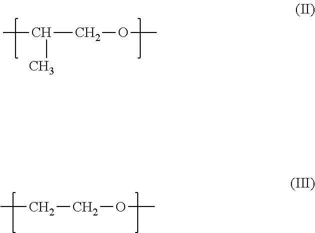

(b) contacting the aqueous solution with a separation membrane, and (c) subjecting the aqueous solution to a diafiltration step to reduce the concentration of soluble low molecular weight components or to introduce one or more soluble low molecular weight components therein and/or to an ultrafiltration step so as to concentrate the protein to produce a retentate product which is an aqueous solution comprising the protein, whereby the compound of formula I reduces aggregation of the protein in method steps (a)-(c) and whereby the compound of formula I passes through the separation membrane in step (c).

The polyalkoxy fatty acyl surfactant of formula I has been found to exhibit a critical micelle concentration (the concentration above which a surfactant spontaneously assembles into micelles) that is lower than that of conventional surfactants such as polysorbates or poloxamer 188, cf. J. S. Katz et al., Mol. Pharmaceutics 2019, 16, pp. 282-291. A lower critical micelle concentration is indicative of a stronger drive to assemble which may translate into a faster stabilization of an interface. Without wishing to be bound by a particular theory, a lower critical micelle concentration may explain the finding that polyalkoxy fatty acyl surfactant of formula I approaches surface equilibrium 1 to 2 orders of magnitude faster than the conventional surfactants and outcompetes a protein at an interface, thus reducing the likelihood of protein aggregation.

The aqueous solution provided in step (a) comprises a protein and a polyalkoxy fatty acyl surfactant of general formula I dissolved therein (e.g., dissolved in water). Optionally the aqueous solution further comprises low molecular weight components such as sugars, sugar alcohols, salts, buffers, amino acids, salts of amino acids, and mixtures thereof. The low molecular weight components are also dissolved in the aqueous solution. When the low molecular weight components are present, preferably the total amount of all low molecular weight components is no more than 300 mg/ml.

Preferred sugars are selected from the group consisting of sucrose, glucose, mannose, trehalose, maltose, dextrose, dextran, and mixtures thereof. Preferred sugar alcohols are selected from the group consisting of sorbitol, mannitol, xylitol, and mixtures thereof. Preferred salts have cations selected from the group consisting of hydrogen, sodium, potassium, magnesium, calcium, ammonium, and mixtures thereof. Preferred salts have anions selected from the group consisting of fluoride, chloride, bromide, iodide, phosphate, carbonate, acetate, citrate, sulfate, and mixtures thereof. Preferred buffers have cations selected from the group consisting of hydrogen, sodium, potassium, magnesium, calcium, ammonium, and mixtures thereof. Preferred amino acids are selected from the group consisting of lysine, glycine, proline, arginine, histidine, and mixtures thereof.

In step (c), the aqueous solution provided in step (a) is subjected to a diafiltration step and/or an ultrafiltration step. In the diafiltration step, at least a portion of the solvent and the polyalkoxy fatty acyl surfactant of formula I pass through the separation membrane. As a result, the generated retentate (aqueous solution comprising protein) comprises a reduced amount or a reduced concentration of the compound of formula I. In the diafiltration step, at least a portion of the low molecular weight components dissolved in the aqueous solution can also pass through the separation membrane. As a result, the generated retentate comprises a reduced amount or a reduced concentration of the low molecular weight components initially present in the aqueous solution provided in step (a). In the diafiltration step, the aqueous solution (e.g., one provided in step (a)) comprising the protein is purified by removing at least a portion of the compound of formula I and undesired low molecular weight components from the aqueous solution.

During diafiltration, the removed solvent and low molecular weight components (filtrate) from the aqueous protein solution (retentate) are replenished with a new solvent. In some embodiments, the new solvent comprises one or more new low molecular weight components dissolved therein, and such one or more new low molecular weight components are introduced into the aqueous protein solution. The new solvent and the one or more new low molecular weight components dissolved therein can independently be same as or different from the solvent and low molecular weight components initially present in the aqueous solution provided in step (a). In some embodiments, at the end of diafiltration, the produced retentate product comprises a reduced concentration of the polyalkoxy fatty acyl surfactant of formula I. In some embodiments, at the end of diafiltration, the concentration of protein in the produced retentate product is substantially same as the protein concentration of the aqueous solution provided in step (a). In some embodiments, at the end of diafiltration, the concentration of protein in the produced retentate product is within the range of ±5%, or ±10%, or ±15% from the protein concentration of the aqueous solution provided in step (a).

In the ultrafiltration step, a portion of the solvent and the polyalkoxy fatty acyl surfactant of formula I passes through the separation membrane. As a result, the concentration of protein in the produced retentate product (aqueous solution comprising protein) is higher than the protein concentration of the aqueous solution provided in step (a), that is, protein in the aqueous solution is concentrated. In some embodiments, the concentration of the polyalkoxy fatty acyl surfactant of formula I in the produced retentate product remains substantially the same comparing with its concentration in the aqueous solution provided in step (a).

In some embodiments, step (c) comprises, consists essentially of, or consists of both a diafiltration step and an ultrafiltration step which are carried out sequentially. In some embodiments, step (c) comprises, consists essentially of, or consists of a diafiltration step followed by an ultrafiltration step, that is, the retentate generated through diafiltration is subject to ultrafiltration. In some embodiments, step (c) comprises, consists essentially of, or consists of an ultrafiltration step, and there is no diafiltration in step (c). In some embodiments, step (c) comprises, consists essentially of, or consists of a diafiltration step, and there is no separate ultrafiltration step in step (c).

The retentate product produced at the end of step (c) is an aqueous solution comprising the protein dissolved therein. In some embodiments, the concentration of the protein in the aqueous solution of the retentate product is from 0.01 mg/ml to 700 mg/ml, preferably from 5 mg/ml to 300 mg/ml. In some embodiments, the aqueous solution of the retentate product is substantially free of the polyalkoxy fatty acyl surfactant of formula I. In some embodiments, the aqueous solution of the retentate product further comprises the polyalkoxy fatty acyl surfactant of formula I dissolved therein. In some embodiments, the concentration of the polyalkoxy fatty acyl surfactant of formula I in the aqueous solution of the retentate product is no more than 1 mg/ml, or no more than 0.1 mg/ml, or no more than 0.05 mg/ml, or no more than 0.01 mg/ml, or no more than 0.005 mg/ml, or no more than 0.001 mg/ml.

In some embodiments, the aqueous solution of the retentate product produced at the end of step (c) comprises at least 80 wt % monomer protein, or at least 85 wt % monomer protein, or at least 90 wt % monomer protein, or at least 92 wt % monomer protein, or at least 94 wt % monomer protein, or at least 96 wt % monomer protein, or at least 98 wt % monomer protein, or at least 99 wt % monomer protein based on the total weight of the protein in the aqueous solution of the retentate product.

In some embodiments, the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In some embodiments, the concentration of the compound of formula I in the aqueous solution provided in step (a) is from about 0.001 mg/ml to about 5 mg/ml, preferably from about 0.005 mg/ml to about 1 mg/ml, preferably from about 0.01 mg/ml to about 0.5 mg/ml, more preferably from about 0.01 mg/ml to about 0.1 mg/ml, more preferably from about 0.01 mg/ml to about 0.05 mg/ml.

In some embodiments, the separation membrane has a molecular weight cut-off of from about 30 kDa to about 50 kDa, the concentration of the compound of formula I in the aqueous solution provided in step a) is from about 0.01 mg/ml to about 0.1 mg/ml or from about 0.01 mg/ml to about 0.05 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In some embodiments, the concentration of the protein in the aqueous solution of step (a) is from 0.0001 mg/ml to 150 mg/ml, preferably from 0.1 mg/ml to 50 mg/ml, more preferably from 1 mg/ml to 20 mg/ml.

In some embodiments, step (c) comprises an ultrafiltration step, and the concentration of the protein in the aqueous solution after ultrafiltration in step c) is from 0.01 mg/ml to 700 mg/ml, preferably from 5 mg/ml to 300 mg/ml.

In some embodiments, the separation membrane has a molecular weight cut-off of from about 10 kDa to about 100 kDa, that is, proteins larger than the molecular weight cut-off can be retained (in the retentate) while smaller proteins and other molecules can pass through the separation membrane to form the filtrate. It is preferred that loss of the protein through the separation membrane is minimized. The separation membrane should therefore be selected to have a molecular weight cut-off not exceeding one third of the molecular weight of the protein to be retained. A molecular weight cut-off in the range of from about 30 kDa to about 50 kDa can serve to retain most large proteins such as antibodies used as biologics. In some embodiments, the separation membrane has a molecular weight cut-off of from about 30 kDa to about 50 kDa.

In some embodiments, the separation membrane has a molecular weight cut-off of about 100 kDa and the concentration of the compound of formula I in the aqueous solution of step (a) is from about 0.005 mg/ml to about 1 mg/ml, or from about 0.05 mg/ml to about 1 mg/ml, or from about 0.01 mg/ml to about 0.5 mg/ml, or from about 0.01 mg/ml to about 0.1 mg/ml, or from about 0.01 mg/ml to about 0.05 mg/ml.

In some embodiments, the separation membrane has a molecular weight cut-off of about 100 kDa, the concentration of the compound of formula I in the aqueous solution of step (a) is from about 0.05 mg/ml to about 1 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In some embodiments, the separation membrane has a molecular weight cut-off of from about 30 kDa to about 50 kDa and the concentration of the compound of formula I in the aqueous solution of step (a) is from about 0.01 mg/ml to about 0.1 mg/ml, or from about 0.01 mg/ml to about 0.05 mg/ml, or from about 0.001 mg/ml to about 0.025 mg/ml, or from about 0.001 mg/ml to about 0.01 mg/ml.

In some embodiments, step (c) comprises, consists essentially of, or consists of an ultrafiltration step and there is no diafiltration preceding or subsequent to the ultrafiltration in step (c), the separation membrane has a molecular weight cut-off of from about 30 kDa to about 50 kDa, and the concentration of the compound of formula I in the aqueous solution of step (a) is from about 0.001 mg/ml to about 0.025 mg/ml, preferably from about 0.001 mg/ml to about 0.01 mg/ml.

In some embodiments, step (c) comprises, consists essentially of, or consists of an ultrafiltration step and there is no diafiltration preceding or subsequent to the ultrafiltration in step (c), the separation membrane has a molecular weight cut-off of from about 30 kDa to about 50 kDa, the concentration of the compound of formula I in the aqueous solution of step (a) is from about 0.001 mg/ml to about 0.025 mg/ml or from about 0.001 mg/ml to about 0.01 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In some embodiments, step (c) comprises, consists essentially of, or consists of a diafiltration step followed by an ultrafiltration step, the separation membrane has a molecular weight cut-off of from about 30 kDa to about 50 kDa, the concentration of the compound of formula I in the aqueous solution of step (a) is from about 0.01 mg/ml to about 0.1 mg/ml or from about 0.01 mg/ml to about 0.05 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In some embodiments, step (c) comprises, consists essentially of, or consists of a diafiltration step and there is no ultrafiltration preceding or subsequent to the diafiltration in step (c), the separation membrane has a molecular weight cut-off of from about 30 kDa to about 50 kDa, and the concentration of the compound of formula I in the aqueous solution of step (a) is from about 0.01 mg/ml to about 0.1 mg/ml.

In some embodiments, step (c) comprises, consists essentially of, or consists of a diafiltration step and there is no ultrafiltration preceding or subsequent to the diafiltration in step (c), the separation membrane has a molecular weight cut-off of from about 30 kDa to about 50 kDa, the concentration of the compound of formula I in the aqueous solution of step (a) is from about 0.01 mg/ml to about 0.1 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

In the polyalkoxy fatty acyl compound of formula I, R¹ is preferably a substituted or unsubstituted aliphatic group. Among substituted aliphatic groups, preferred substituent is hydroxyl. More preferably R¹ is an unsubstituted aliphatic group; more preferably R¹ is an unsubstituted alkyl group. In some embodiments, R¹ is C₉-22 linear alkyl in the compound of formula I, that is, R¹ is a linear alkyl group with 9 to 22 carbon atoms. In some embodiments, R¹ is C₁₀-18 linear alkyl in the compound of formula I. In some embodiments, R¹ is C₁₀-16 linear alkyl in the compound of formula I. In some embodiments, R¹ is C₁₁-15 linear alkyl in the compound of formula I.

Preferably (when n is not 0), X¹ is O or NH. More preferably, X¹ is NH. Preferably, X² is O or NH. More preferably, X² is NH.

n is 0 or 1, 2, 3, 4 or 5. Preferably, n is 0 or 1. More preferably, n is 1.

Preferably, R² has 20 or fewer atoms; more preferably 15 or fewer. Preferably, if R² is not hydrogen, then R² contains one or more carbon atom. Preferably, R² is either hydrogen or an unsubstituted hydrocarbon group; more preferably, R² is either hydrogen, an unsubstituted alkyl group, or an alkyl group whose only substituent is an unsubstituted aromatic hydrocarbon group. Among unsubstituted alkyl groups, preferred is methyl.

Among alkyl groups whose only substituent is an unsubstituted aromatic hydrocarbon group, preferred is —CH₂—(C₆H₅), where —(C₆H₅) is a benzene ring. Preferably, R² represents a side chain of a naturally occurring amino acid. In some embodiments, n is 1 and R² in the compound of formula I represents a side chain of a naturally occurring amino acid. In some embodiments, R² is H, unsubstituted C₁₋₄ alkyl or —CH₂—(C₆H₅) in the compound of formula I.

In some embodiments, R³ has a number-average molecular weight of 600-5000 Daltons, preferably 800-3000 Daltons, in the compound of formula I. In some embodiments, the group R³ is either a statistical copolymer of units of structure (II) and structure (III) or a block copolymer of units of structure (II) and structure (III). Preferably the group R³ is a statistical copolymer of units of structure (II) and structure (III). Preferably, —R³ has the structure —R⁴—CH₃, where R⁴ is a polymeric group comprising, consisting essentially of, or consisting of polymerized units of structure (II) and structure (III). Preferably, R⁴ has no other polymerized units in addition to structure (II) and structure (III).

In some embodiments, R¹ is a linear unsubstituted alkyl group having 10 to 16 carbon atoms, R² is selected from the group consisting of hydrogen, methyl, and —CH₂—(C₆H₅), wherein —(C₆H₅) is a benzene ring, and R³ has number-average molecular weight of from 800 to 3000.

It is useful to characterize the mole ratio (herein the “PO/EO ratio”) of units of structure (II) to units of structure (III). PO is structure (II) and EO is structure (III). In some embodiments, the PO/EO ratio is in the range of from 0.01:1 to 2:1, or from 0.05:1 to 1:1, or from 0.1:1 to 0.5:1.

In a particularly preferred embodiment of the compound of formula (I), R¹ is CH₃—(CH₂)₁₁—CH₂—, n is 1, X¹ and X² are both NH, R² is —CH₂(C₆H₅), and R³ is a copolymer of PO and EO units capped with CH₃ with an approximate number-average molecular weight of 1000 Daltons and ratio of PO to EO of about 3:19. Such compound of formula (I) is denoted as FM1000 in the examples herein.

In another preferred embodiment of the compound of formula (I), R¹ is CH₃—(CH₂)₁₁—CH₂—, n is 0, X² is NH, and R³ is a copolymer of PO and EO units capped with CH₃ with an approximate number-average molecular weight of 1000 Daltons and ratio of PO to EO of about 3:19.

Preferably, the compound of formula (I) has no ionic groups.

The compound of formula (I) may be made by a method disclosed in WO 2017/044366 which is incorporated herein by reference in its entirety for all purposes.

Preferred proteins to be used in the methods of the present disclosure can be selected from the group consisting of monoclonal antibodies, polyclonal antibodies, antibody-drug conjugates, bispecific antibodies, trispecific antibodies, growth factors, insulins, immunoglobulins, peptide hormones, enzymes, polypeptides, fusion proteins, glycosylated proteins, antigens, antigen subunits and combinations thereof.

Preferred proteins have therapeutic efficacy to treat a disease or medical condition or to function as vaccines. Examples of therapeutic proteins are immunoglobulin-g, adalimumab, interferon alfa, bevacizumab, human growth hormone, rituximab, human serum albumin, insulin, erythropoietin alpha, pembrolizumab, etanercept, filgrastim, nivolumab, trastuzumab, durvalumab, interleukin-2, infliximab, chorionic gonadotropin, avelumab, denosumab, ranibizumab, aflibercept, tremelimumab, factor viii, interferon beta, ipilimumab, atezolizumab, abatacept, tocilizumab, ustekinumab, pegfilgrastim, secukinumab, streptokinase, cetuximab, omalizumab, ramucirumab, urokinase, certolizumab pegol, dupilumab, genolimzumab, aldesleukin, molgramostim, peginterferon alfa-2b, tislelizumab, follitropin alfa, gevokizumab, golimumab, spartalizumab, canakinumab, foralumab, varlilumab, nimotuzumab, erythropoietin beta, evolocumab, pegargiminase, bermekimab, carotuximab, daratumumab, eculizumab, ontuxizumab, adalimumab, camrelizumab, enoblituzumab, interleukin-12, lirilumab, panitumumab, gatipotuzumab, relatlimab, andecaliximab, belimumab, cabiralizumab, isactuzumab govitecan, monalizumab, pancreatin, pertuzumab, toripalimab, inebilizumab, ofatumumab, pepinemab, sintilimab, alirocumab, milatuzumab, nidanilimab, sotatercept, vedolizumab, veltuzumab, bevacizumab beta, isatuximab, orlotamab, tisotumab vedotin, benralizumab, cosibelimab, emactuzumab, ganitumab, narsoplimab, pidilizumab, sarilumab, trastuzumab emtansine, anetumab ravtansine, bertilimumab, blinatumomab, guselkumab, ixekizumab, mepolizumab, obinutuzumab, ublituximab, alemtuzumab, emibetuzumab, ficlatuzumab, ifabotuzumab, mirikizumab, natalizumab, racotumomab, siltuximab, timigutuzumab, trastuzumab deruxtecan, bimekizumab, brodalumab, cetrelimab, farletuzumab, opinercept, rilonacept, tomuzotuximab, urelumab, ascrinvacumab, brolucizumab, clazakizumab, cusatuzumab, dalotuzumab, ianalumab, itolizumab, and margetuximab. Also contemplated are proteins that can be used as medical diagnostics or have a beneficial effect on a food composition, or can be incorporated in a cleaning composition or a coatings formulation.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

### Materials

Unless otherwise noted, all materials were received from Sigma-Aldrich or Fisher and used without further purification. Jeffamine M-1000 was provided by Huntsman. Polysorbate 80 (PS80) was the “tested according to Ph.Eur.” grade, Sigma-Aldrich product number 59924. It was stored under a nitrogen headspace to minimize oxidation. Industrial grade bovine IgG (immunoglobulin G) was purchased from MP Biomedicals (Santa Ana, Calif.).

FM1000 is a surfactant compound of formula (I), wherein R¹ is CH₃—(CH₂)₁₁—CH₂—, n is 1, X¹ and X² are both NH, R² is —CH₂—(C₆H₅), where —(C₆H₅) is a benzene ring, and R³ is a copolymer of PO and EO units capped with CH₃ with an approximate molecular weight of 1000 Daltons and PO/EO ratio of about 3:19. FM1000 was prepared as reported in WO 2017/044366. Briefly, myristoyl chloride was amidated with phenylalanine in water in the presence of sodium hydroxide and triethylamine. The resulting suspension was acidified to pH 2 with concentrated HCl and filtered. The filtered powder was then recrystallized from hexanes. The myristoyl phenylalanine was amidated by melt condensation with Jeffamine M-1000. The crude FM1000 product was dissolved in methanol and stirred over DuPont Amberlite™ IRN 77 and IRN78 ion exchange resins to remove starting materials. The final product was dried under vacuum.

### Example 1: Retention of FM1000 During Centrifugal Filtration

1 mg/ml solutions of FM1000 in deionized water or 0.9% saline were prepared. Half of the samples were removed directly from a fridge while the other half were briefly heated to 60° C. before centrifugation. 2 ml of each solution were placed into Amicon Ultra-4 centrifuge tubes and were centrifuged for 2 min at 3000 rpm. Molecular weight cut-off (MWCO) of the filters (separation membranes) was 30, 50, or 100 kDa. The filtrate was measured for FM1000 concentration by HPLC (high-performance liquid chromatography) analysis. Results are shown in Table 1.

TABLE 1
Concentration of FM1000 in filtrate, 100 kDa MWCO filter

| | Thermal History | | | |
|---|---|---|---|---|
| | 4° C. | | 60° C. | |
| | Solution | | | |
| | 0.9% Saline | Water | 0.9% Saline | Water |
| FM1000 | 0.83 | 0.80 | 0.89 | 0.85 |
| Concentration (mg/ml) | | | | |

There was no detectable FM1000 in the filtrate passed through 30 or 50 kDa MWCO filters under any of the test conditions. These data showed that FM1000, at concentration of 1 mg/ml, can efficiently pass through 100 kDa MWCO separation membrane, but cannot efficiently pass through 30 or 50 kDa MWCO separation membrane.

### Examples 2-5

For Examples 2-5, a 200 ml aqueous solution comprising IgG at the concentration of 1 mg/ml and FM1000 was prepared in the reservoir of a standard ultrafiltration (UF) device. For diafiltration (DF), the main reservoir contained the same aqueous solution (comprising IgG at the concentration of 1 mg/ml and FM1000) as the one in the ultrafiltration reservoir. The second reservoir contained the replacement aqueous solution which was same as the one in the main reservoir except that the replacement aqueous solution did not have FM1000 and IgG in it. The replacement aqueous solution was drawn into the main reservoir at the same rate that the aqueous solution was filtered out to waste, that is, the replacement aqueous solution was drawn into the main reservoir at the same rate that the filtrate was formed. In this way, the concentration of protein in the aqueous solution was maintained essentially constant during diafiltration. All aliquots for analysis were collected from the ultrafiltration reservoir or the diafiltration main reservoir. Filters (separation membranes) were Pall Minimate Capsules with 30 kDa MWCO, and flow rate was set to 120 ml/min.

### Example 2 (Comparative): UF of an Aqueous Solution Comprising 0.05 mg/ml FM1000 and 1 mg/ml IgG

A 200 ml aqueous solution (0.9% saline) comprising IgG at the concentration of 1 mg/ml and FM1000 at the concentration of 0.05 mg/ml was subjected to UF only and was concentrated to about 5 ml (retentate volume). The separation membrane MWCO was 30 kDa. Samples were collected at various points of concentration (i.e., various points of retentate volume) and were analyzed by UV/Vis (A280 nm, 10× dilution) for IgG concentration and HPLC (A210 nm) for FM1000 concentration. Results are shown in Table 2.

TABLE 2

| Retentate Volume | HPLC | UV/Vis |
|---|---|---|
| 200 ml | 162.3 | 0.066 |
| (Initial, t = 0) | | |

| | | | |
|---|---|---|---|
| 150 | ml | 136.9 | 0.082 |
| 100 | ml | 176.7 | 0.104 |
| 50 | ml | 262.1 | 0.170 |
| 25 | ml | 657.0 | 0.397 |
| 5 | ml | 758.1 | 0.506 |

These data showed that FM1000 at the concentration of 0.05 mg/ml cannot efficiently pass through a 30 kDa MWCO separation membrane in an ultrafiltration process.

### Example 3: DF/UF of an Aqueous Solution Comprising 0.05 mg/ml FM1000 and 1 Mg/Ml IgG

In Example 3, a 200 ml aqueous solution comprising IgG at the concentration of 1 mg/ml and FM1000 at the concentration of 0.05 mg/ml was subjected to DF and then UF, that is, the aqueous solution of the retentate product produced at the end of DF was subjected to UF. The 200 ml aqueous solution (subjected to DF) was either a 0.9% saline solution or a 25 mM (millimolar) histidine buffer solution at pH 6.5. When the 200 ml aqueous solution (subjected to DF) was the 0.9% saline solution (comprising IgG and FM1000), the replacement aqueous solution in the second reservoir was pure 0.9% saline solution without IgG and FM1000. When the 200 ml aqueous solution (subjected to DF) was the 25 mM histidine buffer solution at pH 6.5 (comprising IgG and FM1000), the replacement aqueous solution in the second reservoir was pure 25 mM histidine buffer solution at pH 6.5 without IgG and FM1000.

During DF, the 200 ml 0.9% saline solution (comprising IgG and FM1000) in the main reservoir was diafiltration exchanged with 1.6 liter pure 0.9% saline solution (without IgG and FM1000), and the 200 ml 25 mM histidine buffer solution at pH 6.5 (comprising IgG and FM1000) was diafiltration exchanged with 1.0 liter pure 25 mM histidine buffer solution at pH 6.5 (without IgG and FM1000). The respective aqueous solution of the retentate product produced at the end of DF was further subjected to UF to reduce the volume of retentate to about 25 ml so that protein IgG contained therein was concentrated.

The separation membranes used for DF and UF in Example 3 had MWCO of 30 kDa. Samples were collected at various points of DF and UF process and were analyzed by UV/Vis (A280 nm, 10× dilution) for IgG concentration and HPLC (A210 nm) for FM1000 concentration. Results are shown in Table 3, where column “Saline HPLC” shows HPLC signal strength (indicating FM1000 concentration) at various points of DF and UF of the 0.9% saline solution, column “Saline UV/Vis” shows UV/Vis signal strength (indicating IgG concentration) at various points of DF and UF of the 0.9% saline solution, column “Histidine HPLC” shows HPLC signal strength at various points of DF and UF of the 25 mM histidine buffer solution, and column “Histidine UV/Vis” shows UV/Vis signal strength (indicating IgG concentration) at various points of DF and UF of the 25 mM histidine buffer solution.

TABLE 3

| | Saline | Saline | Histidine | Histidine |
|---|---|---|---|---|
| DF/UF Progress | HPLC | UV/Vis | HPLC | UV/Vis |
| Initial Solution (t = 0) | 46.23 | 0.105 | 101.85 | 0.114 |
| 1 liter DF exchange | 19.39 | 0.095 | 10.14 | 0.116 |
| 1.6 liter DF exchange | 16.53 | 0.094 | — | — |
| 50 ml Retentate | 15.81 | 0.210 | 22.39 | 0.256 |
| Volume (UF) | | | | |
| 25 ml Retentate | 6.92 | 0.333 | 46.92 | 0.369 |
| Volume (UF) | | | | |

These data showed that FM1000 at the concentration of 0.05 mg/ml can be efficiently removed during a process comprising a diafiltration step followed by an ultrafiltration step where 30 kDa MWCO separation membranes are used for both DF and UF. These data also showed that FM1000 at the concentration of 0.05 mg/ml can efficiently pass through a 30 kDa MWCO separation membrane in a diafiltration process.

### Example 4: DF/UF of an Aqueous Solution Comprising 0.1 mg/ml FM1000 and 1 Mg/Ml IgG

In Example 4, a 200 ml 0.9% saline solution comprising IgG at the concentration of 1 mg/ml and FM1000 at the concentration of 0.1 mg/ml was subjected to DF and then UF. The DF/UF process was conducted in the same manner as in Example 3 except the FM1000 concentration in the initial 200 ml aqueous solution was 0.1 mg/ml, only 0.9% saline solution was used/tested in the process and diafiltration exchanged with 1.5 liter pure 0.9% saline solution (without IgG and FM1000) in DF, and UF reduced the volume of retentate to about 50 ml. The separation membranes used for DF and UF in Example 4 had MWCO of 30 kDa. Results are shown in Table 4.

TABLE 4

| DF/UF Progress | HPLC | UV/Vis |
|---|---|---|
| Initial Solution (t = 0) | 352.3 | 0.0681 |
| 1 liter DF exchange | 129.5 | 0.0718 |
| 1.5 liter DF exchange | 74.4 | 0.0716 |
| 100 ml Retentate | 196.5 | 0.1162 |
| Volume (UF) | | |
| 50 ml Retentate | 507.8 | 0.2149 |
| Volume (UF) | | |

These data showed that FM1000 at the concentration of 0.1 mg/ml can be slowly and partially diafiltered out of the aqueous solution (retentate) using 30 kDa MWCO separation membrane. The subsequent ultrafiltration with 30 kDa MWCO separation membrane concentrated both IgG and FM1000, but the FM1000/protein concentration ratio in the 50 ml aqueous solution of the retentate product was much smaller than one in the initial 200 ml 0.9% saline solution.

### Example 5: UF of an Aqueous Solution Comprising 0.01 mg/ml FM1000 and 1 mg/ml IgG

A 200 ml aqueous solution (0.9% saline) comprising IgG at the concentration of 1 mg/ml and FM1000 at the concentration of 0.01 mg/ml was subjected to UF only and was concentrated to about 2 ml (retentate volume). The separation membrane MWCO was 30 kDa. Samples were collected at various points of concentration (i.e., various points of retentate volume) and were analyzed by UV/Vis (A280 nm, 10× dilution) for IgG concentration and HPLC (A210 nm) for FM1000 concentration. Results are shown in Table 5.

TABLE 5

| Retentate Volume | HPLC | UV/Vis |
|---|---|---|
| 200 ml | <LLD | 0.0664 |
| (Initial, t = 0) | | |

| | | | |
|---|---|---|---|
| 100 | ml | <LLD | 0.1116 |
| 50 | ml | <LLD | 0.2146 |
| 25 | ml | <LLD | 0.3321 |
| 2 | ml | <LLD | 1.0836 |

<LLD means below the lower limit of detection. These data showed that protein IgG was concentrated. However, no FM1000 was ever detected, indicating that it was not concentrated during UF. Therefore, these data showed that FM1000 at the concentration of 0.01 mg/ml can efficiently pass through a 30 kDa MWCO separation membrane in an ultrafiltration process.

### Example 6: Effect of FM1000 and Polysorbate 80 (Both at the Concentration of 0.01 Mg/Ml) on Stability of Protein Cetuximab in Aqueous Solution

Cetuximab was commercially available under the trade name Erbitux® and was acquired from a pharmacy. It was formulated as a 2 mg/ml aqueous solution in 10 mM phosphate buffer and 145 mM sodium chloride, pH 7.2.

Erbitux® solutions (2 mg/ml cetuximab) were reformulated to add FM1000 or polysorbate 80 into the solutions and achieve target cetuximab concentrations. The 1.5 mg/ml cetuximab samples were prepared by diluting Erbitux® solutions. The 7.5 mg/ml cetuximab samples were prepared by concentrating Erbitux® solutions via centrifugal filtration to 10 mg/ml and then diluting to 7.5 mg/ml. The surfactant (FM1000 or polysorbate 80) concentration in the cetuximab samples was 0.01 mg/ml. In samples 9 and 10, no surfactant was added. The cetuximab samples were either shaken (post-shake) or not shaken (pre-shake) before analysis. Each shaken sample was 3.0 ml in a 5 ml serum vial from Wheaton. Pre-shake samples were aliquots removed to bring the shaken sample volume to 3.0 ml. Shaken samples were shaken overnight at 150 strokes/min on a reciprocal shaker.

Particles (indicating protein cetuximab aggregates) present in the sample solutions were analyzed, including the amount of particles (particle count) and the particle size (expressed as radius of particles). Results are shown in Table 6. Microflow imaging was performed on a Biotechne Microflow Imaging unit to quantify subvisible particles. Particle counts were automated by the software. Size exclusion chromatography was run on an Agilent 1260 Bioinert liquid chromatography system with UV detection. The column was an Agilent AdvanceBio 300A SEC column and running buffer was phosphate buffer. UV absorbance was measured on a Molecular Devices M3 Plate reader. Dynamic Light Scattering (DSC) was measured on a Wyatt DynaPro II.

TABLE 6

| | CTX | Surf | | | | |
|---|---|---|---|---|---|---|
| Samp | Conc | Conc | | | Radius | Particle |
| # | (mg/ml) | (mg/ml) | Surf | Condition | (nm) | Count |
| 1 | 1.5 | 0.01 | FM1000 | Pre-Shake | 5.8 | |
| 2 | 1.5 | 0.01 | PS80 | Pre-Shake | 5.5 | |
| 3 | 1.5 | 0.01 | FM1000 | Post-Shake | 5.7 | 3803 |
| 4 | 1.5 | 0.01 | PS80 | Post-Shake | 8.9 | 29418 |
| 5 | 7.5 | 0.01 | FM1000 | Pre-Shake | 6.3 | |
| 6 | 7.5 | 0.01 | PS80 | Pre-Shake | 6.3 | |
| 7 | 7.5 | 0.01 | FM1000 | Post-Shake | 6.3 | 777 |
| 8 | 7.5 | 0.01 | PS80 | Post-Shake | 6.8 | 3874 |
| 9 | 7.5 | 0 | none | Pre-Shake | | 861 |
| 10 | 7.5 | 0 | none | Post-Shake | | 628344 |

In Table 6, Samp means sample, CTX means cetuximab, Conc means concentration, and Surf means surfactant. These data showed that at a level of 0.01 mg/ml surfactant, FM1000 was capable to reduce aggregation of the protein cetuximab much more significantly than polysorbate 80.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

### EMBODIMENTS

For further illustration, additional non-limiting embodiments of the present disclosure are set forth below.

For example, embodiment 1 is a method of stabilizing a protein in aqueous solution during purification and/or concentration of the protein. The method comprises the steps of: (a) providing an aqueous solution comprising a protein and a polyalkoxy fatty acyl surfactant of general formula I

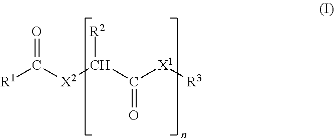

wherein R¹—C(═O) is a fatty acyl group, R² is H or a substituted or unsubstituted hydrocarbyl group, X¹ is S, O or NH, X² is S, O or NH, n is 0 or an integer of 1-5, R³ is a polymeric group comprising polymerized units of general formula II and III

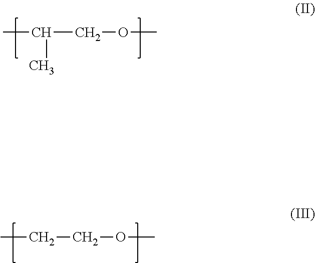

(b) contacting the aqueous solution with a separation membrane, and (c) subjecting the aqueous solution to a diafiltration step to reduce the concentration of soluble low molecular weight components or to introduce one or more soluble low molecular weight components therein and/or to an ultrafiltration step so as to concentrate the protein to produce a retentate product which is an aqueous solution comprising the protein, whereby the compound of formula I reduces aggregation of the protein in method steps (a)-(c) and whereby the compound of formula I passes through the separation membrane in step (c).

Embodiment 2 is a method as set forth in embodiment 1 wherein the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 3 is a method as set forth in any of the preceding embodiments, wherein step (c) comprises both a diafiltration step and an ultrafiltration step carried out sequentially.

Embodiment 4 is a method as set forth in any of the preceding embodiments, wherein the concentration of the compound of formula I in the aqueous solution provided in step (a) is from about 0.001 mg/ml to about 5 mg/ml, preferably from about 0.005 mg/ml to about 1 mg/ml, preferably from about 0.01 mg/ml to about 0.5 mg/ml, more preferably from about 0.01 mg/ml to about 0.1 mg/ml, more preferably from about 0.01 mg/ml to about 0.05 mg/ml.

Embodiment 5 is a method as set forth in any of the preceding embodiments, wherein the separation membrane has a molecular weight cut-off of from about 30 kDa to about 50 kDa, the concentration of the compound of formula I in the aqueous solution provided in step a) is from about 0.01 mg/ml to about 0.1 mg/ml or from about 0.01 mg/ml to about 0.05 mg/ml, the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 6 is a method as set forth in any of the preceding embodiments, wherein the concentration of the protein in the aqueous solution of step (a) is from 0.0001 mg/ml to 150 mg/ml, preferably from 0.1 mg/ml to 50 mg/ml, more preferably from 1 mg/ml to 20 mg/ml.

Embodiment 7 is a method as set forth in any of the preceding embodiments, wherein step (c) comprises an ultrafiltration step, and the concentration of the protein in the aqueous solution after ultrafiltration in step c) is from 0.01 mg/ml to 700 mg/ml, preferably from 5 mg/ml to 300 mg/ml.

Embodiment 8 is a method as set forth in any one of embodiments 1-4 and 6-7, wherein the separation membrane has a molecular weight cut-off of from about 10 kDa to about 100 kDa, preferably from about 30 kDa to about 50 kDa.

Embodiment 9 is a method as set forth in any one of embodiments 1-4 and 6-8, wherein the separation membrane has a molecular weight cut-off of about 100 kDa and wherein the concentration of the compound of formula I in the aqueous solution of step (a) is from about 0.005 mg/ml to about 1 mg/ml, or from about 0.05 mg/ml to about 1 mg/ml, or from about 0.01 mg/ml to about 0.5 mg/ml, or from about 0.01 mg/ml to about 0.1 mg/ml, or from about 0.01 mg/ml to about 0.05 mg/ml.

Embodiment 10 is a method as set forth in embodiment 9 wherein the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 11 is a method as set forth in any one of embodiments 1-3 and 6-7, wherein the separation membrane has a molecular weight cut-off of from about 30 kDa to about 50 kDa and wherein the concentration of the compound of formula I in the aqueous solution of step (a) is from about 0.01 mg/ml to about 0.1 mg/ml.

Embodiment 12 is a method as set forth in any one of embodiments 1-2 and 6, wherein step (c) comprises, consists essentially of, or consists of an ultrafiltration step and there is no diafiltration preceding or subsequent to the ultrafiltration in step (c), the separation membrane has a molecular weight cut-off of from about 30 kDa to about 50 kDa, and the concentration of the compound of formula I in the aqueous solution of step (a) is from about 0.001 mg/ml to about 0.025 mg/ml, preferably from about 0.001 mg/ml to about 0.01 mg/ml.

Embodiment 13 is a method as set forth in embodiment 12, wherein the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 14 is a method as set forth in any one of embodiments 1-2 and 6, wherein step (c) comprises, consists essentially of, or consists of a diafiltration step followed by an ultrafiltration step, the separation membrane has a molecular weight cut-off of from about 30 kDa to about 50 kDa, and the concentration of the compound of formula I in the aqueous solution of step (a) is from about 0.01 mg/ml to about 0.1 mg/ml or from about 0.01 mg/ml to about 0.05 mg/ml.

Embodiment 15 is a method as set forth in embodiment 14, wherein the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 16 is a method as set forth in any one of embodiments 1-2 and 6, wherein step (c) comprises, consists essentially of, or consists of a diafiltration step and there is no ultrafiltration preceding or subsequent to the diafiltration in step (c), the separation membrane has a molecular weight cut-off of from about 30 kDa to about 50 kDa, and the concentration of the compound of formula I in the aqueous solution of step (a) is from about 0.01 mg/ml to about 0.1 mg/ml.

Embodiment 17 is a method as set forth in embodiment 16, wherein the aqueous solution of the retentate product comprises at least 80 wt %, or at least 85 wt %, or at least 90 wt %, or at least 95 wt % monomer protein based on the total weight of the protein in the aqueous solution of the retentate product, and the surfactant/protein concentration ratio in the aqueous solution of the retentate product produced at the end of step (c) is reduced by at least 50%, or at least 60%, or at least 70%, or at least 80%, or at least 85%, or at least 90%, or at least 95%, or at least 97%, or at least 99% comparing with the surfactant/protein concentration ratio in the aqueous solution provided in step (a).

Embodiment 18 is a method as set forth in any of the preceding embodiments, wherein R¹ is C₉₋₂₂ linear alkyl in the compound of formula I.

Embodiment 19 is a method as set forth in embodiment 18, wherein R¹ is C₁₁₋₁₅ linear alkyl in the compound of formula I.

Embodiment 20 is a method as set forth in any of the preceding embodiments, wherein X² is NH in the compound of formula I.

Embodiment 21 is a method as set forth in any of the preceding embodiments, wherein n is 1, 2, 3, 4 or 5, preferably 1, in the compound of formula I.

Embodiment 22 is a method as set forth in any of the preceding embodiments, wherein X¹ is NH in the compound of formula I.

Embodiment 23 is a method as set forth in any of the preceding embodiments, wherein n is 1 and R² in the compound of formula I represents a side chain of a naturally occurring amino acid.

Embodiment 24 is a method as set forth in any one of embodiments 1-22, wherein R² is H, unsubstituted C₁₋₄ alkyl or —CH₂—(C₆H₅) in the compound of formula I.

Embodiment 25 is a method as set forth in any of the preceding embodiments, wherein R³ has a number-average molecular weight of 600-5000 Daltons, preferably 800-3000 Daltons, in the compound of formula I.

Embodiment 26 is a method as set forth in any one of embodiments 1-17 and 20-22, wherein, in the compound of formula I, R¹ is a linear unsubstituted alkyl group having 10 to 16 carbon atoms, R² is selected from the group consisting of hydrogen, methyl, and —CH₂—(C₆H₅), wherein —(C₆H₅) is a benzene ring, and R³ has number-average molecular weight of from 800 to 3000.

Embodiment 27 is a method as set forth in any of the preceding embodiments, wherein the ratio of propylene oxide (PO) units to ethylene oxide (EO) units is in the range of from 0.01:1 to 2:1, preferably from 0.05:1 to 1:1, more preferably from 0.1:1 to 0.5:1, in the compound of formula I.

Embodiment 28 is a method as set forth in any one of embodiments 1-17, wherein, in the compound of formula I, R¹ is CH₃—(CH₂)₁₁—CH₂—, n is 1, X¹ and X² are both NH, R² is —CH₂(C₆H₅), and R³ is a copolymer of PO and EO units capped with CH₃ with a number-average molecular weight of about 1000 Daltons and ratio of PO to EO of about 3:19.

Embodiment 29 is a method as set forth in any one of embodiments 1-17, wherein, in the compound of formula I, R¹ is CH₃—(CH₂)₁₁—CH₂—, n is 0, X² is NH, and R³ is a copolymer of PO and EO units capped with CH₃ with a number-average molecular weight of about 1000 Daltons and ratio of PO to EO of about 3:19.

Embodiment 30 is a method as set forth in any of the preceding embodiments, wherein the protein is selected from the group consisting of monoclonal antibodies, polyclonal antibodies, antibody-drug conjugates, bispecific antibodies, trispecific antibodies, growth factors, insulins, immunoglobulins, peptide hormones, enzymes, polypeptides, fusion proteins, glycosylated proteins, antigens, antigen subunits, and combinations thereof.

Embodiment 31 is a method as set forth in any of the preceding embodiments, wherein the aqueous solution of step (a) further comprises a sugar selected from the group consisting of sucrose, glucose, mannose, trehalose, maltose, dextrose, dextran, and mixtures thereof.

Embodiment 32 is a method as set forth in any one of embodiments 1-30, wherein the aqueous solution of step (a) further comprises a sugar alcohol selected from the group consisting of sorbitol, mannitol, xylitol, and mixtures thereof.

Embodiment 33 is a method as set forth in any of the preceding embodiments, wherein the aqueous solution of step (a) further comprises a salt, the salt has cation selected from the group consisting of hydrogen, sodium, potassium, magnesium, calcium, ammonium, and mixtures thereof, and the salt has anion selected from the group consisting of fluoride, chloride, bromide, iodide, phosphate, carbonate, acetate, citrate, sulfate, and mixtures thereof.

Embodiment 34 is a method as set forth in any of the preceding embodiments, wherein the aqueous solution of step (a) further comprises a naturally occurring amino acid.

Embodiment 35 is a method as set forth in embodiment 34, wherein the amino acid is selected from the group consisting of lysine, glycine, proline, arginine, histidine, and mixtures thereof.

1. A method of stabilizing a protein in aqueous solution during purification and/or concentration of the protein, the method comprising the steps of:
(a) providing an aqueous solution comprising a protein and a polyalkoxy fatty acyl surfactant of general formula I: 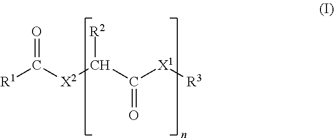
wherein:
R¹—C(═O) is a fatty acyl group,
R² is H or a substituted or unsubstituted hydrocarbyl group,
X¹ is S, O or NH, X² is S, O or NH,
n is 0 or an integer of 1-5,
R³ is a polymeric group comprising polymerized units of general formula II and III: 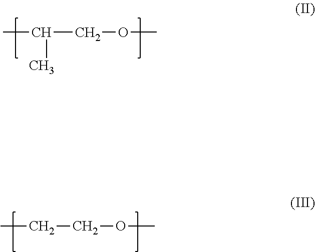
(b) contacting the aqueous solution with a separation membrane, and
(c) subjecting the aqueous solution;
to a diafiltration step to reduce the concentration of soluble low molecular weight components or to introduce one or more soluble low molecular weight components therein, and/or
to an ultrafiltration step so as to concentrate the protein to produce a retentate product which is an aqueous solution comprising the protein, whereby the compound of formula I reduces aggregation of the protein in method steps (a)-(c) and whereby the compound of formula I passes through the separation membrane in step (c).

2. The method of claim 1, wherein the surfactant/protein concentration ratio in the aqueous solution of the retentate product produced at the end of step (c) is reduced by at least 50% comparing with the surfactant/protein concentration ratio in the aqueous solution provided in step (a).

3. The method of claim 1, wherein the concentration of the compound of formula I in the aqueous solution provided in step (a) is from about 0.001 mg/ml to about 5 mg/ml.

4. The method of claim 1, wherein:
the separation membrane has a molecular weight cut-off of about 100 kDa, and
the concentration of the compound of formula I in the aqueous solution of step (a) is from about 0.005 mg/ml to about 1 mg/ml.

5. The method of claim 1, wherein:
the separation membrane has a molecular weight cut-off of from about 30 kDa to about 50 kDa, and
the concentration of the compound of formula I in the aqueous solution of step (a) is from about 0.01 mg/ml to about 0.1 mg/ml.

6. The method of claim 1, wherein:
step (c) comprises an ultrafiltration step and there is no diafiltration preceding or subsequent to the ultrafiltration in step (c),
the separation membrane has a molecular weight cut-off of from about 30 kDa to about 50 kDa, and
the concentration of the compound of formula I in the aqueous solution of step (a) is from about 0.001 mg/ml to about 0.025 mg/ml.

7. The method of claim 1, wherein:
step (c) comprises a diafiltration step followed by an ultrafiltration step,
the separation membrane has a molecular weight cut-off of from about 30 kDa to about 50 kDa, and
the concentration of the compound of formula I in the aqueous solution of step (a) is from about 0.01 mg/ml to about 0.1 mg/ml.

8. The method of claim 1, wherein:
step (c) comprises a diafiltration step and there is no ultrafiltration preceding or subsequent to the diafiltration in step (c),
the separation membrane has a molecular weight cut-off of from about 30 kDa to about 50 kDa, and
the concentration of the compound of formula I in the aqueous solution of step (a) is from about 0.01 mg/ml to about 0.1 mg/ml.

9. The method of claim 1, wherein both X¹ and X² are NH.

10. The method of claim 1, wherein n is 1.

11. The method of claim 1, wherein:
R¹ is a linear unsubstituted alkyl group having 10 to 16 carbon atoms;
R² is selected from the group consisting of hydrogen, methyl, and —CH₂—(C₆H₅), wherein —(C₆H₅) is a benzene ring; and
R³ has number-average molecular weight of from 800 to 3000.

12. The method of claim 1, wherein:
R¹ is CH₃—(CH₂)₁₁—CH₂—,
n is 1, X¹ and X² are both NH,
R² is —CH₂(C₆H₅), and
R³ is a copolymer of PO and EO units capped with CH₃ with a number-average molecular weight of about 1000 Daltons and ratio of PO to EO of about 3:19.

13. The method of claim 1, wherein:
R¹ is CH₃—(CH₂)₁₁—CH₂—,
n is 0, X² is NH, and
R³ is a copolymer of PO and EO units capped with CH₃ with a number-average molecular weight of about 1000 Daltons and ratio of PO to EO of about 3:19.
